(19) United States
(12) Patent Application Publication
(10) Pub. No.: US 2012/0010691 A1
(43) Pub. Date: Jan. 12, 2012

(54) Particle Embedded Polymer Stent and Method of Manufacture

(75) Inventor: Gerry Clarke, Co. Galway (IE)

(73) Assignee: Medtronic Vascular, Inc., Santa Rosa, CA (US)

(21) Appl. No.: 12/830,582

(22) Filed: Jul. 6, 2010

(51) Int. Cl. A61F 2/84; B05D 3/12; A61F 2/82

(52) U.S. Cl. 623/1.11; 623/1.42; 427/2.28

(57) ABSTRACT

A particle embedded polymer stent and method of manufacture, which includes a stent delivery system having a catheter; a balloon operably attached to the catheter; and a polymer stent disposed on the balloon, the stent comprising struts interconnected to form a tubular body. Each of the struts includes in cross section a drug-free core region; and a drug region surrounding and immediately adjacent to the core region, the drug region including drug particles. The drug-free core region and the drug region are made of a single polymer, the single polymer having a drug-safe softening temperature.

### TECHNICAL FIELD

The technical field of this disclosure is medical implant devices, particularly, particle embedded polymer stents and method of manufacture.

### BACKGROUND OF THE INVENTION

Stents are generally cylindrical shaped devices that are radially expandable to hold open a segment of a blood vessel or other anatomical lumen after implantation into the body lumen. Stents have been developed with coatings to deliver drugs or other therapeutic agents.

Stents are used in conjunction with balloon catheters in a variety of medical therapeutic applications including intravascular angioplasty. For example, a balloon catheter device is inflated during PTCA (percutaneous transluminal coronary angioplasty) to dilate a stenotic blood vessel. The stenosis may be the result of a lesion such as a plaque or thrombus. After inflation, the pressurized balloon exerts a compressive force on the lesion thereby increasing the inner diameter of the affected vessel. The increased interior vessel diameter facilitates improved blood flow. Soon after the procedure, however, a significant proportion of treated vessels re-narrow.

To prevent restenosis, short flexible cylinders, or stents, constructed of metal or various polymers are implanted within the vessel to maintain lumen size. The stent acts as a scaffold to support the lumen in an open position. Various configurations of stents include a cylindrical tube defined by a mesh, interconnected stents or like segments. Some exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz and U.S. Pat. No. 5,421,955 to Lau. Balloon-expandable stents are mounted on a collapsed balloon at a diameter smaller than when the stents are deployed. Stents can also be self-expanding, growing to a final diameter when deployed without mechanical assistance from a balloon or like device.

Drug eluting stents currently employ exterior coatings with or without polymers on metal struts to hold a drug for subsequent elution and delivery of the drug to surrounding tissue. Unfortunately, such coatings present a number of problems and limitations. The coatings are fragile and can fracture and fragment during manufacture, delivery, deployment, or use. Fracture during manufacture increases the cost and complexity of manufacture. Fracture during delivery, deployment, or use can reduce the effectiveness of the stent due to lost drug and can pose a risk to the patient if fragments block blood flow.

The manufacture of coated drug eluting stents presents additional problems. The atomized spraying of the coating onto the metal framework is inefficient and wastes costly drugs. The drug and polymer is typically mixed with a solvent for spraying, so strict environmental controls are required to protect manufacturing personnel from the volatile spray. Such environmental controls increase the cost and complexity of manufacture.

One proposed solution has been to make the whole stent from a polymer material and mix the drug with the polymer feed for injection molding into the final stent. Unfortunately, the temperatures achieved in plasticization of the polymer destroy the effectiveness of most drugs of interest. Another proposed solution has been to electrostatically spray the drug onto the stent with an adhesive to attach the drug to the stent surface, but most drugs of interest are non-conductive and cannot be sprayed electrostatically.

It would be desirable to have a particle embedded polymer stent and method of manufacture that would overcome the above disadvantages.

### SUMMARY OF THE INVENTION

One aspect of the invention provides a stent delivery system including a catheter; a balloon operably attached to the catheter; and a polymer stent disposed on the balloon, the stent comprising struts interconnected to form a tubular body. Each of the struts includes in cross section a drug-free core region; and a drug region surrounding and immediately adjacent to the core region, the drug region including drug particles. The drug-free core region and the drug region are made of a single polymer, the single polymer having a drug-safe softening temperature.

Another aspect of the invention provides a stent including struts interconnected to form a tubular body. Each of the struts includes in cross section a drug-free core region; and a drug region surrounding and immediately adjacent to the core region, the drug region including drug particles. The drug-free core region and the drug region are made of a single polymer, the single polymer having a drug-safe softening temperature.

Another aspect of the invention provides a method of manufacturing a polymer stent including providing a polymer stent blank having struts interconnected to form a tubular body, each of the struts in cross section having a softened region surrounding and immediately adjacent to a core region, the softened region being at a drug-safe softening temperature, the softened region and the core region being made of a single polymer; depositing drug particles into the softened regions; and cooling the softened regions including the drug particles to form drug regions, the polymer stent having finished struts, each of the finished struts in cross section having the drug region surrounding and immediately adjacent to the core region

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 is a perspective view of a stent delivery system made in accordance with the invention.

FIG. 2 is a side view of a polymer stent made in accordance with the invention.

FIG. 3 is a cross section view of a strut of a polymer stent made in accordance with the invention.

FIGS. 4A & 4B are detail cross section views of a strut of a polymer stent made in accordance with the invention.

FIG. 5 is a flow chart of a method of manufacture of a polymer stent in accordance with the invention.

FIGS. 6A-6C are various views of a method of manufacture of a polymer stent in accordance with the invention.

FIG. 7 is a block diagram of a system for manufacture of a polymer stent in accordance with the invention.

### DETAILED DESCRIPTION

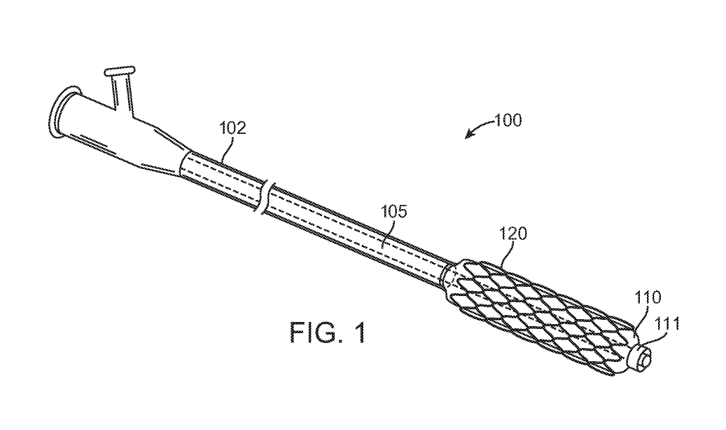

FIG. 1 is a perspective view of a stent delivery system made in accordance with the invention. The stent delivery system 100 includes a catheter 105, a balloon 110 operably attached to the catheter 105, and a polymer stent 120 disposed on the balloon 110. The polymer stent 120 is operable for use in a vessel having a vessel wall forming a vessel lumen.

The balloon 110, shown in an inflated state, can be any variety of balloons capable of expanding the polymer stent 120. The balloon 110 can be manufactured from a material such as polyethylene, polyethylene terephthalate (PET), nylon, Pebax® polyether-block co-polyamide polymers, or the like. In one embodiment, the stent delivery system 100 can include retention means 111, such as mechanical or adhesive structures, for retaining the polymer stent 120 on the balloon 110 until the polymer stent 120 is deployed. The catheter 105 may be any variety of balloon catheter, such as a PTCA (percutaneous transluminal coronary angioplasty) balloon catheter, capable of supporting a balloon during angioplasty. The stent delivery system 100 can also include a sheath 102 through which the polymer stent 120 can be delivered to the deployment site.

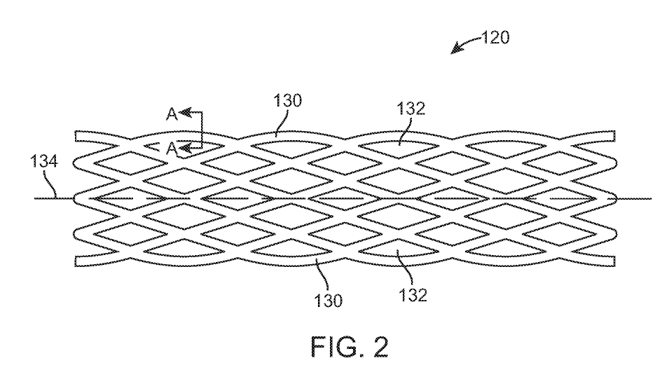

FIG. 2 is a side view of a polymer stent made in accordance with the invention. The polymer stent includes a number of struts interconnected to form a tubular body. Each of the struts in cross section has a drug-free core region and a drug region, including drug particles, disposed about the core region. The drug-free core region and a drug region are made of the same polymer, which has a drug-safe softening temperature. The polymer stent 120 can be installed in the stent delivery system of FIG. 1 for implantation in a body lumen, such as a vessel lumen.

Referring to FIG. 2, the polymer stent 120 includes a number of struts 130 interconnected to form the tubular body of the polymer stent 120. The struts 130 in cross section have a drug-free core region and a drug region, including drug particles, disposed about the core region. The polymer stent 120 includes at least one opening 132 and has a central axis 134 with openings 132 generally perpendicular to the central axis. The pattern of the struts 130 can be W-shaped or can be a more complex shape with the elements of one segment continuing into the adjacent segment. In one embodiment, the polymer stent 120 can be expanded by a balloon or another device. In another embodiment, the polymer stent 120 can be self-expanding. In one embodiment, a top coat (not shown) can be disposed on the struts 130.

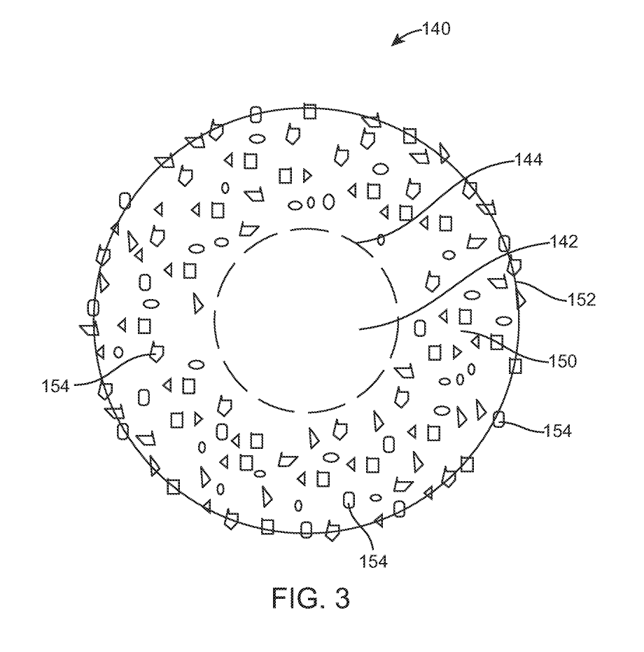

FIG. 3 is a cross section view of a strut of a polymer stent made in accordance with the invention. The strut has in cross section a drug-free core region, and a drug region surrounding and immediately adjacent to the core region. The drug region includes drug particles. The drug-free core region and the drug region are made of a single polymer, which has a drug-safe softening temperature. The cross section view is taken at Section A-A of FIG. 2.

Referring to FIG. 3, which is a cross section view of a strut, the strut 140 includes a drug-free core region 142 and a drug region 150. The drug-free core region has a region boundary 144 and the drug region 150 has an outer surface 152. Those skilled in the art will appreciate that the region boundary 144 is not necessarily an absolute boundary, but can be determined by a marked change in the concentration of the drug particles at the region boundary 144. The drug region 150 includes drug particles 154, which can be partially embedded in the outer surface 152 or completely embedded within the drug region 150. In this example, some of the drug particles 154 are partially embedded and some of the drug particles 154 are completely embedded. The drug particles 154 can be irregularly shaped drug particles or smooth shaped drug particles. In this example, the drug particles 154 are a mixture of irregularly shaped drug particles and smooth shaped drug particles. In another example, all the drug particles 154 can be of one shape.

The single polymer making up the drug-free core region and the drug region has a drug-safe softening temperature, i.e., the polymer softens at a temperature that is low enough to allow deposition of the drug particles 154 without reducing the effectiveness of the drug in the drug particles 154. The thickness of the drug region, i.e., the thickness between the region boundary 144 and the outer surface 152, is defined by depth of penetration of the drug particles 154 into a softened region of a polymer stent blank. The drug region 150 is formed from the softened region of the polymer stent blank. Those skilled in the art will appreciate that the thickness of the drug region can be selected as desired for a particular application. The drug elution profile and duration depends on the distribution of the drug particles 154 in the drug region 150. In one example, the diameter of the strut 140 is 0.004 inches and the thickness of the drug region 150 is 0.0008 inches In another example, the diameter of the strut 140 is 0.004 inches and the thickness of the drug region 150 is 0.0002 inches. In one example, the drug particles 154 have a diameter in the range of 0.00001 to 0.001.

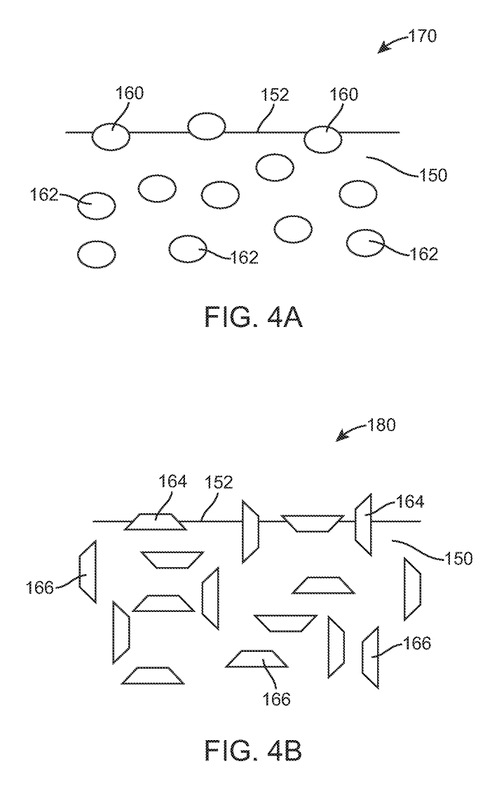

FIGS. 4A & 4B, in which like elements share like reference numbers with FIG. 3, are detail cross section views of a strut of a polymer stent made in accordance with the invention.

Referring to FIG. 4A, which is a cross section view of a strut with smooth drug particles, the strut 170 includes smooth shaped drug particles 160, 162. The smooth shaped drug particles 160 are partially embedded in the outer surface 152 and the smooth shaped drug particles 162 are completely embedded within the drug region 150. The term smooth shaped drug particles as used herein is defined as drug particles that are substantially free of external points, such as spheres, spheroids, ellipsoids, or the like. The smooth shaped drug particles need not be symmetrical about a single axis, but can be multi-lobed and/or asymmetric. In this example, the strut 170 includes both partially embedded and completely embedded drug particles. In one embodiment, the strut can include partially embedded drug particles alone. The partially embedded drug particles can have different exposure above the outer surface 152 and different depths of penetration into the drug region as desired for a particular application. In another embodiment, the strut can include completely embedded drug particles alone. The completely embedded drug particles can have different depths of penetration into the drug region as desired for a particular application.

Referring to FIG. 4B, which is a cross section view of a strut with irregularly shaped drug particles, the strut 180 includes irregularly shaped drug particles 164, 166. The irregularly shaped drug particles 164 are partially embedded in the outer surface 152 and the irregularly shaped drug particles 166 are completely embedded within the drug region 150. The term irregularly shaped drug particles as used herein is defined as drug particles that have any number of external points, such as trapezoidal solids, regular polyhedra, general prisms, or the like. The irregularly shaped drug particles need not be symmetrical, but can be crystal shaped, multi-legged, multifaceted, and/or asymmetric. In this example, the strut 180 includes both partially embedded and completely embedded drug particles. In one embodiment, the strut can include partially embedded drug particles alone. The partially embedded drug particles can have different exposure above the outer surface 152 and different depths of penetration into the drug region as desired for a particular application. The irregular shape can help retain the partially embedded drug particles in the outer surface 152. In another embodiment, the strut can include completely embedded drug particles alone. The completely embedded drug particles can have different depths of penetration into the drug region as desired for a particular application.

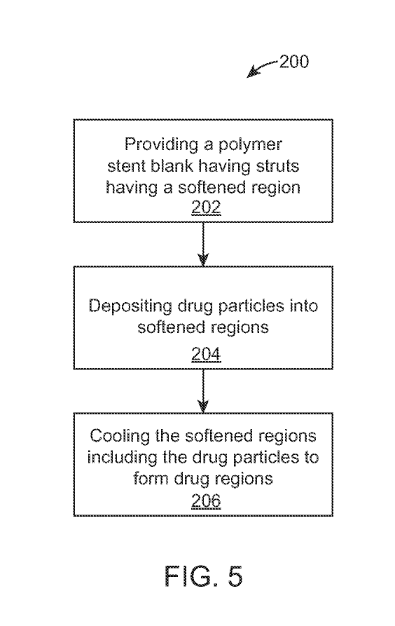

FIG. 5 is a flow chart of a method of manufacture of a polymer stent in accordance with the invention. The method 200 includes providing a polymer stent blank having struts interconnected to form a tubular body 202, each of the struts in cross section having a softened region surrounding and immediately adjacent to a core region, the softened region being at a drug-safe softening temperature, the softened region and the core region being made of a single polymer; depositing drug particles into the softened regions 204; and cooling the softened regions including the drug particles to form drug regions 206, the polymer stent having finished struts, each of the finished struts in cross section having the drug region surrounding and immediately adjacent to the core region.

In one embodiment, the providing 202 can include delivering the polymer stent blank directly from an injection mold machine producing the polymer stent blank through an injection molding process, where the softened region of the polymer stent blank is at a drug-safe softening temperature from the injection molding process. In one embodiment, the polymer stent blank is delivered directly from the injection mold machine into a temperature controlled enclosure. The temperature controlled enclosure can be used to maintain the desired temperature when depositing drug particles and/or cooling the softened regions. The drug particles can be deposited into the softened regions within the temperature controlled enclosure.

In another embodiment, the providing 202 can include heating the softened region of the polymer stent blank to the drug-safe softening temperature. The polymer stent blank can be heated to the drug-safe softening temperature from ambient temperature or another temperature below the drug-safe softening temperature. The heating can be performed in a controlled environment by any desired indirect heating method, such as convective heating, radiant heating, or the like. The heating can be used to heat the polymer stent blank to form a predetermined thickness for the softened regions, which can be used to determine how far the drug particles are deposited into the polymer stent blank and the thickness of the drug region in the final stent, which is formed from the softened region.

The drug particles can be deposited 204 into the softened regions in various manners. The drug particles can be irregularly shaped drug particles or smooth shaped drug particles. In one embodiment, the drug particles are deposited into the softened regions with a gas jet including drug particles and an appropriate inert gas. The gas jet can be moved about a stationary polymer stent blank, or the polymer stent blank can be moved axially and rotated on a mandrel with the gas jet held in a stationary position. The velocity of the gas jet can be used to determine how far the drug particles are deposited into the polymer stent blank. The temperature of the gas jet can be used to maintain the softened regions of the polymer stent blank at the desired temperature. This application technique can be referred to as bombardment, i.e., spraying solids with no solvent onto the softened outer surface of the stent.

In another embodiment, the drug particles are deposited 204 into the softened regions by rolling the polymer stent blank in the drug particles. The polymer stent blank can be placed in a bed of drug particles which is agitated to ensure even coverage of the polymer stent blank with the drug particles. Rolling the polymer stent blank in the drug particles can be used to encrust the outer surface of the polymer stent blank with the drug particles, with the drug particles partially embedded in the outer surface. In yet another embodiment, the drug particles are deposited into the softened regions by suspending the polymer stent blank in a fluidized bed of the drug particles. The polymer stent blank and the drug particles are both suspended in the fluidized bed and the drug particles deposited into the softened regions. The temperature of the fluidized bed can be used to maintain the softened regions of the polymer stent blank at the desired temperature. Those skilled in the art will appreciate that the drug particles can be deposited on one or more polymer stent blanks simultaneously as desired. The drug particles can be deposited into the softened regions to partially embed the drug particles in the outer surface of the polymer stent blank or to completely embed the drug particles within the softened region.

The cooling of the softened regions including the drug particles to form drug regions 206 can be performed at a controlled rate to obtain desired characteristics in the drug regions. The formation of the drug regions fixes the drug particles into the outer surface of the polymer stent when drug particles are partially embedded in the outer surface of the polymer stent blank.

The method 200 can further include applying a top coat onto the drug regions before or after the polymer stent blank has cooled. Those skilled in the art will appreciate that the top coat can be applied by spraying, painting, rolling, electrostatic deposition, ink jet coating, spin coating, or the like of a polymer and solvent onto the drug regions as desired for a particular application. In one embodiment, the top coat can be applied by bombardment, i.e., spraying solids with no solvent onto the softened outer drug regions of the stent.

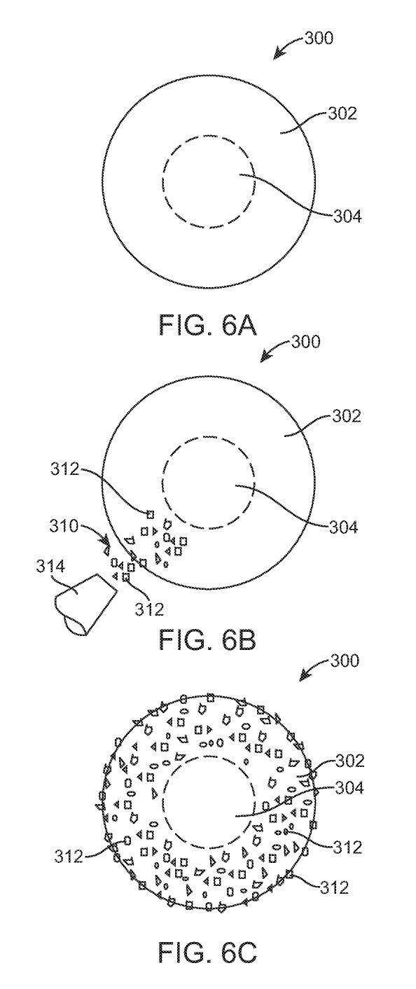

FIGS. 6A-6C, in which like elements share like reference numbers, are various views of a method of manufacture of a polymer stent in accordance with the invention. In this example, the drug particles are deposited into the softened regions with a gas jet.

FIG. 6A is a cross section view of strut of a polymer stent blank for use in a method of manufacture of a polymer stent in accordance with the invention. The polymer stent blank 300 includes a softened region 302 surrounding and immediately adjacent to a core region 304, the softened region 302 being at a drug-safe softening temperature. The softened region 302 and the core region 304 are made of a single polymer. The thickness and temperature of the softened region 302 can be selected as desired for a particular application. Those skilled in the art will appreciate that the thickness of the softened region 302 can be thin when the drug particles are to be partially embedded in the outer surface of the softened region 302 alone.

The single polymer forming the softened region 302 and the core region 304 can be any polymer with a drug-safe softening temperature, which as used herein is defined as a temperature at which the single polymer softens enough to allow deposition of the drug particles without reducing the effectiveness of the drug in the drug particles. Those skilled in the art will appreciate that the value of the drug-safe softening temperature depends on the particular drug which is to be used in the polymer stent. At the drug-safe softening temperature, the polymer is soft and the outer surface can be tacky, but the polymer is not molten. In one example, the drug-safe softening temperature is in the range of 50 to 75 degrees Celsius. In another example, the drug-safe softening temperature is at or below the Vicat softening point, which is the temperature at which a thermoplastic material reaches a specific degree of softness, as measured by a standardized indentation test. The single polymer hardens to a polymer having desired properties for a particular application, such as strength, flexibility, and the like, at a deployment/use temperature that is less than the drug-safe softening temperature.

The single polymer can be any polymer having a drug-safe softening temperature and being compatible with a selected drug or therapeutic agent. In one example, the single polymer is high density polyethylene, which has a softening temperature of about 65 degrees Celsius. In another example, the polymer is a member of the polyolefin family. Other exemplary polymers include polymers such as BioLinx® polymer, poly(vinyl alcohol), poly(ethylene-vinyl acetate), polyurethane, polycaprolactone, polyglycolide, poly(lactide-co-glycolide), poly(ethylene oxide), poly(vinyl pyrrolidone), silicone, an acrylic polymer, an acrylic and acrylonitrile copolymer, a latex polymer, a thermoplastic polymer, a thermoset polymer, a biostable polymer, a biodegradable polymer, a blended polymer, a copolymer, combinations thereof, and the like. Those skilled in the art will appreciate that the various polymers and combinations of polymers with a particular drug-safe softening temperature can be used as desired for a particular application.

FIG. 6B is a cross section view of a polymer stent blank during drug particle deposition in a method of manufacture of a polymer stent in accordance with the invention. In this example, a jet 310 of drug particles and gas is ejected from a nozzle 314 to deposit the drug particles 312 into the softened region 302. Those skilled in the art will appreciate that the drug particles can be deposited by other methods, such as rolling the polymer stent blank in drug particles, suspending the polymer stent blank in a fluidized bed of drug particles, or the like.

The drug particles 312 can include any drug compatible with the single polymer of the softened region 302 which maintains its efficacy at the drug-safe softening temperature. The term drugs as used herein is defined as any drug, therapeutic agent, bioactive agent, or the like intended to affect the structure or any function of the body of man or other animals. In one example, the drug is the synthetic analog of rapamycin ABT-578, which maintains efficacy at about 65 degrees Celsius. Other exemplary drugs, therapeutic agents, or bioactive agents include drugs such as an antirestenotic drug (e.g., rapamycin, rapamycin analogue, or rapamycin derivative to prevent or reduce the recurrence or narrowing and blockage of the bodily vessel), an anti-cancer drug (e.g., camptothecin or other topoisomerase inhibitors), an antisense agent, an antineoplastic agent, an antiproliferative agent, an antithrombogenic agent, an anticoagulant, an antiplatelet agent, an antibiotic, an anti-inflammatory agent, a steroid, a gene therapy agent, an organic drug, a pharmaceutical compound, a recombinant DNA product, a recombinant RNA product, a collagen, a collagenic derivative, a protein, a protein analog, a saccharide, a saccharide derivative, a bioactive agent, a pharmaceutical drug, a therapeutic substance, a combination thereof, and the like.

FIG. 6C is a cross section view of a polymer stent blank during cooling for use in a method of manufacture of a polymer stent in accordance with the invention. The polymer stent blank 300 becomes the finished polymer stent, with the softened region 302 including the drug particles 312 becoming the drug region, and the core region 304 becoming the drug-free core region. The softened region 302 can be cooled at a cooling rate that provides the desired properties in the polymer stent, such as strength or flexibility. A top coat can be applied to the outer surface of the polymer stent blank 300 before or after cooling as desired for a particular application.

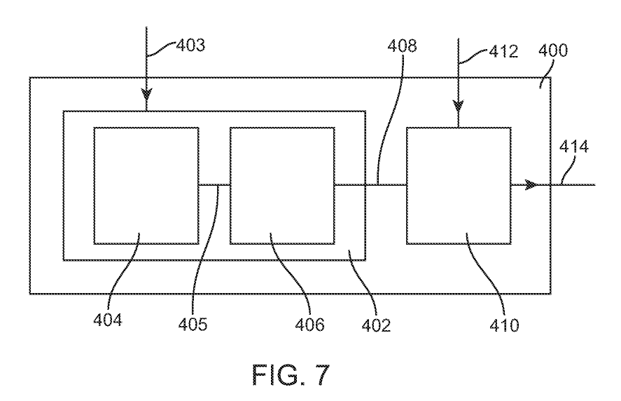

FIG. 7 is a block diagram of a system for manufacture of a polymer stent in accordance with the invention. The system provides a compact, sealed, unitary package for receiving polymer feed stock and producing a finished polymer stent. The system 400 includes a micro molding machine 402 and a particle deposition chamber 410. The micro molding machine 402 has an extruder portion 404 and a molding portion 406. Exemplary micro molding machines include the Battenfeld Microsystem 50, available from Wittman-Battenfeld Inc, of Torrington, Conn. In one embodiment, the particle deposition chamber 410 can be a temperature controlled enclosure.

In operation, the extruder portion 404 receives and plasticizes a polymer feed stock 403 to generate molten polymer 405, which is provided to the molding portion 406. The molding portion 406 molds the molten polymer 405 into a polymer stent blank 408, which is provided to the particle deposition chamber 410. The particle deposition chamber 410 also receives drug particles 412 for deposition on the polymer stent blank 408.

In one embodiment, the particle deposition chamber 410 can be a temperature controlled enclosure. When the polymer stent blank 408 received at the particle deposition chamber 410 has a softened region at a drug-safe softening temperature, no temperature correction is required, and the drug particle deposition can proceed. Otherwise, the particle deposition chamber 410 can heat (with convective or radiant heating) or cool the polymer stent blank 408 until the polymer stent blank 408 has a softened region at a drug-safe softening temperature for the drug particles 412. The particle deposition chamber 410 can deposit the drug particles 412 into the softened regions of the polymer stent blank 408 by injection of the drug particles 412 with an air jet, rolling the polymer stent blank 408 in the drug particles 412, suspending the polymer stent blank 408 in a fluidized bed of the drug particles 412, or the like. The particle deposition chamber 410 can include suitable manual or automatic handling devices to move the polymer stent blank 408 into and within the particle deposition chamber 410, and to move the polymer stent 414 out of the particle deposition chamber 410. In one embodiment, the particle deposition chamber 410 can cool the polymer stent at a predetermined rate before the polymer stent 414 exits the particle deposition chamber 410. In one embodiment, the particle deposition chamber 410 can apply a top coat to the polymer stent before the polymer stent exits the particle deposition chamber 410.

It is important to note that FIGS. 1-7 illustrate specific applications and embodiments of the invention, and are not intended to limit the scope of the present disclosure or claims to that which is presented therein. Upon reading the specification and reviewing the drawings hereof, it will become immediately obvious to those skilled in the art that myriad other embodiments of the invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A stent delivery system comprising:
a catheter;
a balloon operably attached to the catheter; and
a polymer stent disposed on the balloon, the stent comprising struts interconnected to form a tubular body, each of the struts comprising in cross section:
a drug-free core region; and
a drug region surrounding and immediately adjacent to the core region, the drug region including drug particles;
wherein the drug-free core region and the drug region are made of a single polymer, the single polymer having a drug-safe softening temperature.

2. The system of claim 1 wherein the thickness of the drug region is defined by depth of penetration of the drug particles into a softened region of a polymer stent blank.

3. The system of claim 1 wherein the drug region has an outer surface and the drug particles are partially embedded in the outer surface.

4. The system of claim 3 wherein the drug particles are irregularly shaped drug particles.

5. The system of claim 1 wherein the drug particles are completely embedded within the drug region.

6. The system of claim 1 wherein the drug particles are irregularly shaped drug particles.

7. The system of claim 1 wherein the drug particles are smooth shaped drug particles.

8. The system of claim 1 further comprising a top coat surrounding and immediately adjacent to the drug region.

9. A polymer stent comprising:
struts interconnected to form a tubular body, each of the struts comprising in cross section:
a drug-free core region; and
a drug region surrounding and immediately adjacent to the core region, the drug region including drug particles;
wherein the drug-free core region and the drug region are made of a single polymer, the single polymer having a drug-safe softening temperature.

10. The stent of claim 9 wherein the thickness of the drug region is defined by depth of penetration of the drug particles into a softened region of a polymer stent blank.

11. The stent of claim 9 wherein the drug region has an outer surface and the drug particles are partially embedded in the outer surface.

12. The stent of claim 11 wherein the drug particles are irregularly shaped drug particles.

13. The stent of claim 9 wherein the drug particles are completely embedded within the drug region.

14. The stent of claim 9 wherein the drug particles are irregularly shaped drug particles.

15. The stent of claim 9 wherein the drug particles are smooth shaped drug particles.

16. The stent of claim 9 further comprising a top coat surrounding and immediately adjacent to the drug region.

17. The stent of claim 9 wherein the drug-safe softening temperature is between 50 and 75 degrees Celsius.

18. A method of manufacturing a polymer stent comprising:
providing a polymer stent blank having struts interconnected to form a tubular body, each of the struts in cross section having a softened region surrounding and immediately adjacent to a core region, the softened region being at a drug-safe softening temperature, the softened region and the core region being made of a single polymer;
depositing drug particles into the softened regions; and
cooling the softened regions including the drug particles to form drug regions, the polymer stent having finished struts, each of the finished struts in cross section having the drug region surrounding and immediately adjacent to the core region.

19. The method of claim 18 wherein the providing comprises delivering the polymer stent blank directly from an injection mold machine producing the polymer stent blank through an injection molding process, the softened region of the polymer stent blank being at the drug-safe softening temperature from the injection molding process.

20. The method of claim 19 wherein the delivering the polymer stent blank directly from an injection mold machine comprises delivering the polymer stent blank into a temperature controlled enclosure.

21. The method of claim 20 wherein the depositing comprises depositing drug particles into the softened regions within the temperature controlled enclosure.

22. The method of claim 18 wherein the providing comprises heating the softened region of the polymer stent blank to the drug-safe softening temperature.

23. The method of claim 22 wherein the heating is performed by a heating method selected from convective heating and radiant heating.

24. The method of claim 22 wherein the heating comprises heating the polymer stent blank to form a predetermined thickness for the softened regions.

25. The method of claim 18 wherein the depositing comprises injecting the drug particles into the softened region with a gas jet.

26. The method of claim 18 wherein the depositing comprises rolling the polymer stent blank in the drug particles.

27. The method of claim 18 wherein the depositing comprises suspending the polymer stent blank in a fluidized bed of the drug particles.

28. The method of claim 18 wherein the softened region has an outer surface and the depositing comprises partially embedding the drug particles in the outer surface.

29. The method of claim 18 wherein the depositing comprises completely embedding the drug particles within the softened region.

30. The method of claim 18 wherein the drug particles are irregularly shaped drug particles.

31. The method of claim 18 wherein the drug particles are smooth shaped drug particles.

32. The method of claim 18 further comprising applying a top coat onto the drug regions.

33. The method of claim 18 wherein the drug-safe softening temperature is between 50 and 75 degrees Celsius.
